(19) United States
(12) Patent Application Publication
(10) Pub. No.: US 2024/0390869 A1
(43) Pub. Date: Nov. 28, 2024

(54) METHODS OF CONTROLLING THE LEVEL OF DISSOLVED OXYGEN (DO) IN A SOLUTION COMPRISING A RECOMBINANT PROTEIN IN A STORAGE CONTAINER

(71) Applicant: BRISTOL-MYERS SQUIBB COMPANY, Princeton, NJ (US)

(72) Inventors: Danielle BELLUSCIO, Oxford, MA (US); Derek CHOY, Hudson, MA (US); Akshay NEELI, Jamaica Plain, MA (US)

(73) Assignee: BRISTOL-MYERS SQUIBB COMPANY, Princeton, NJ (US)

(21) Appl. No.: 18/693,731

(22) PCT Filed: Sep. 20, 2022
(86) PCT No.: PCT/US2022/076684

§ 371 Date: Mar. 20, 2024

Related U.S. Application Data
(60) Provisional application No. 63/246,385, filed on Sep. 21, 2021.

(51) Int. Cl. B01F 25/54; B01F 23/234; B01F 25/53; C07K 1/14

(52) U.S. Cl. CPC B01F 25/54; B01F 23/23413; B01F 25/53; C07K 1/14

(57) ABSTRACT

This invention provides a method of controlling the level of dissolved oxygen in a solution comprising a recombinant protein in a storage container.

### CROSS-REFERENCE TO RELATED APPLICATIONS

This application claims priority to U.S. Provisional Application Ser. No. 63/246,385, filed Sep. 21, 2021, the entire contents of which is incorporated herein by reference.

### FIELD OF THE INVENTION

The present invention generally relates to methods of controlling the level of dissolved oxygen in a solution comprising a recombinant protein in a storage container.

### BACKGROUND OF THE INVENTION

Protein therapeutics which include monoclonal antibodies become more popular in treatment of multiple human diseases. Protein bears certain heterogeneity due to a wide range of post-translational modifications. Successful control of therapeutic protein manufacturing process is critical for ensuring product quality, safety, and lot to lot consistency. In fact, the ICH Q6B guideline requires a product appearance specification.

During the manufacturing process, reduction of the disulfide bond and oxidation of methionine residues of the recombinant proteins have been widely observed in the harvest and downstream purification and they could result in protein degradation. Dissolved oxygen (DO) control is an important component in mitigating reduction of mAbs and oxidation of other proteins. Multiple process parameters have impacts on the extent of antibody reduction. For example, maintaining high levels of dissolved oxygen during harvest is vital to keep antibody molecules intact (Mun et al., Biotechnol Bioeng 112 (4): 734-42 (2015); Trexler-Schmidt et al., Biotechnol Bioeng 15; 106 (3): 452-61 (2010)). Several strategies such as sparger method (U.S. Pat. No. 8,574,869, FIG. 1) and dynamic overlay method (WO 2018/200430, FIG. 2) have been developed to control DO during the harvest storage. One of its major limitations for the sparger method is limited accessibility for implementation in downstream product-hold vessels, such as Single Use Mixers (SUM). SUMs are used as product hold vessels for intermediate process pools in downstream processing such as post-harvest clarified bulk, protein A chromatography eluate, AEX/CEX chromatography load/eluate, etc.

Therefore, the need exists for improved methods of producing therapeutic protein specially antibodies that reduce or eliminate degradation and chemical modification, including unwanted disulfide bond reduction by controlling dissolved oxygen during the harvest and the downstream process.

### BRIEF SUMMARY OF THE INVENTION

Here, we introduce a reverse sparger method which can be implemented in SUM vessels of varied sizes and enable efficient dissolved oxygen control (FIG. 3).

This invention discloses a method of controlling the level of a dissolved oxygen in a solution which comprises a recombinant protein in a storage container, comprising recirculating the solution by drawing the solution to the air overlay of the top of the container.

In certain aspects of the invention, the recirculating is through a tube.

In certain aspects of the invention, the recirculation is driven by a pump.

In certain aspects of the invention, the recirculated solution is directed to the liquid surface or the interior wall of the container.

In certain aspects of the invention, the top end of the tube is connected to a nozzle.

In certain aspects of the invention, the nozzle is curved.

In certain aspects of the invention, the nozzle is a J nozzle, J cutaway nozzle, a double J nozzle, or a double J cutaway nozzle.

In certain aspects of the invention, the air overlay space comprises greater than 1% of the interior volume of the container.

In certain aspects of the invention, the method further comprises refreshing the air overlay with new air or a new gas through inlet and outlet ports in the container.

In certain aspects of the invention, the new gas is oxygen.

In certain aspects of the invention, the new gas is nitrogen, argon, or carbon dioxide.

In certain aspects of the invention, the new gas is nitrogen.

In certain aspects of the invention, the storage container is a bag or a tank.

In certain aspects of the invention, the recombinant protein is a recombinant disulfide-bond containing protein.

In certain aspects of the invention, the recombinant protein is a monoclonal antibody.

In certain aspects of the invention, the monoclonal antibody binds an antigen selected from the group consisting of PD-1, PD-L1, CTLA-4, LAG-3, TIGIT, GITR, CXCR4, CD73, HER2, VEGF, CD20, CD40, CD11a, tissue factor (TF), PSCA, IL-8, IL-13, EGFR, SARS-COV-2 spike protein, HER3, and HER4.

In certain aspects of the invention, the recombinant protein is a Fc-fusion protein.

In certain aspects of the invention, the solution is clarified harvest or partially purified protein solution.

### BRIEF DESCRIPTION OF THE DRAWINGS

FIG. 1 shows a depiction of the existing technology for dissolved oxygen control with sparger method.

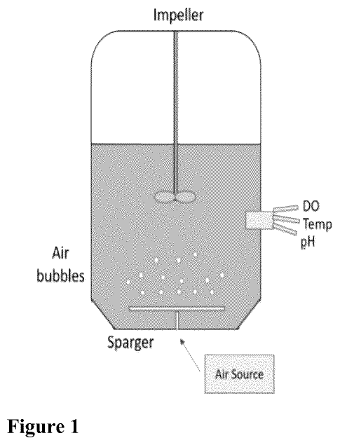

FIG. 2 shows a depiction of the existing technology for dissolved oxygen control with dynamic overlay method.

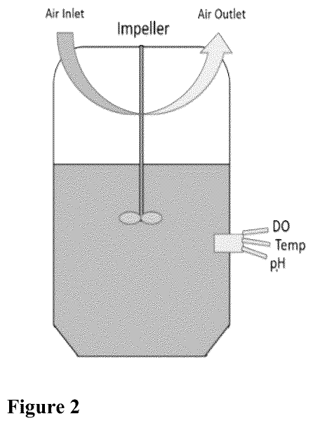

FIG. 3 shows a depiction of the current invention for dissolved oxygen control with reverse sparger method.

FIG. 4 shows examples of nozzle types that can be used in the reverse sparger method.

FIG. 5 shows comparison of oxygen transfer rates between the reverse sparger and the air overlay method. Static air consisted of a closed bag with no air inlets. Dissolved oxygen levels were measured for 4 hours starting at 0% DO.

FIG. 6 shows chart comparing the time taken to reduce dissolved oxygen from 100% to 0% in the reverse sparger and the air overlay.

FIG. 7 shows comparison of K_{L}a values of the reverse sparger with that of the open pipe sparger and the dynamic air overlay

FIG. 8 shows example of a graph demonstrating the gassing out method. Point A to B represent the nitrogen reducing the dissolved oxygen levels. Point B is when the oxygen is turned back on and point C represents the dissolved oxygen saturation point.

### DETAILED DESCRIPTION OF THE INVENTION

The invention discloses a method of controlling the level of a dissolved oxygen in a solution which comprises a recombinant protein in a storage container, comprising recirculating the solution by drawing the solution to the air overlay of the top of the container.

### Definitions

In order that the present disclosure may be more readily understood, certain terms are first defined. As used in this application, except as otherwise expressly provided herein, each of the following terms shall have the meaning set forth below. Additional definitions are set forth throughout the application.

The indefinite articles “a” or “an” should be understood to refer to “one or more” of any recited or enumerated component.

The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean a range of plus or minus 50% of a stated reference value, preferably a range of plus or minus 25%, or more preferably a range of plus or minus 10%. When particular values or compositions are provided in the application and claims, unless otherwise stated, the meaning of “about” should be assumed to be within an acceptable error range for that particular value or composition.

The term “protein” as used herein refers to a peptide-linked chain of amino acids regardless of length. One or more amino acid residues in the protein may contain a modification such as, but not limited to, glycosylation, phosphorylation, or disulfide bond formation. The term “protein” is used interchangeably herein with “polypeptide.”

The term “recombinant protein” as used herein refers to a protein expressed from a host cell (e.g., a mammalian host cell) that has been genetically engineered to express that protein. The recombinantly expressed protein can be identical or similar to protein that is normally expressed in the host cell. The recombinantly expressed protein can also be foreign to the host cell, i.e. heterologous to peptides normally expressed in the host cell.

An “antibody” (Ab) shall include, without limitation, a glycoprotein immunoglobulin (Ig) which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region of an IgG Ab comprises three constant domains, CH1, CH2 and CH3. Each light chain comprises a light chain variable region (abbreviated herein as VL) and a light chain constant region. The light chain constant region of an IgG Ab comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1-CDR1-FR2-CDR2-FR3-CDR3-FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

An Ig may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM, IgG1, or IgG4) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized partially or fully by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

The term “monoclonal antibody” (mAb) as used herein refers to a non-naturally occurring preparation of Ab molecules of single molecular composition, i.e., Ab molecules whose primary sequences are essentially identical, which exhibits a single binding specificity and affinity for a particular epitope. A mAb is an example of an isolated Ab. mAbs may be produced by hybridoma, recombinant, transgenic or other techniques known to those skilled in the art.

In certain aspects of the invention, the recombinant protein is a monoclonal antibody. In certain aspects of the invention, the monoclonal antibody is a chimeric antibody, a humanized antibody, or a human antibody.

A “chimeric” Ab refers to an Ab in which the variable regions are derived from one species and the constant regions are derived from another species, such as an Ab in which the variable regions are derived from a mouse Ab and the constant regions are derived from a human Ab.

A “human” mAb (HuMAb) as used herein refers to a mAb having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human” Ab, as used herein, is not intended to include Abs in which CDR sequences derived from the germline of another mammalian species, such as a mouse, have been grafted onto human framework sequences. The terms “human” Abs and “fully human” Abs are used synonymously.

A “humanized” mAb as used herein refers to a mAb in which some, most or all of the amino acids outside the CDR domains of a non-human mAb are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

The term “disulfide bond” as used herein refers to the covalent bond between two sulfur atoms. In proteins, disulfide bonds between two cysteine residues often contribute to the protein's three-dimensional structure and stability.

In certain aspects of the invention, the recombinant protein contains at least one pair of disulfide bond.

The term “disulfide-bond containing protein” as used herein refers to the protein which contains at least one pair of disulfide bond.

In certain aspects of the invention, the recombinant protein is a recombinant disulfide-bond containing protein.

The term “disulfide bond reduction” as used herein refers to the chemical process by which a sulfur-sulfur bond is broken and replaced by a hydrogen-sulfur bond. The interconversion of disulfide groups (characterized by an S—S bond) and thiols (characterized by an H—S bond) represents a redox reaction, wherein the thiol represents the reduced state and the disulfide represents the oxidized state. In proteins, disulfide linkages between two cysteine residues often contribute to the protein's three-dimensional structure and stability; thus, the reduction of disulfide linkages can result in the formation of low molecular weight protein species. The extent of disulfide bond reduction and the presence of low molecular weight species can be measured by known methods, such as capillary electrophoresis. Such measurements are often presented as the % monomer in a sample.

An “Fc region” (fragment crystallizable region), “Fc domain” or “Fc” as used herein refers to the C-terminal region of the heavy chain of an Ab that mediates the binding of the Ig to host tissues or factors, including binding to FcRs located on various cells of the immune system (e.g., effector cells) or to the first component (C1q) of the classical complement system. Thus, the Fc region is a polypeptide comprising the constant region of an Ab excluding the first constant region Ig domain. In IgG, IgA and IgD Ab isotypes, the Fc region is composed of two identical protein fragments, derived from the second (CH2) and third (CH3) constant domains of the Ab's two heavy chains; IgM and IgE Fc regions contain three heavy chain constant domains (CH domains 2-4) in each polypeptide chain. For IgG, the Fc region comprises Ig domains Cγ2 and Cγ3 and the hinge between Cγ1 and Cγ2. As used herein, the Fc region may be a native sequence Fc or a variant Fc.

The term “Fc-fusion protein”, as used herein, is meant to encompass therapeutic proteins comprising an Fc domain moiety and a moiety derived from a second, non-immunoglobulin protein.

In certain aspects of the invention, the recombinant protein is a Fc-fusion protein.

As used herein, the term “tube” refers to a form of container that can compress in order to dispense the contents through an orifice provided at one end thereof.

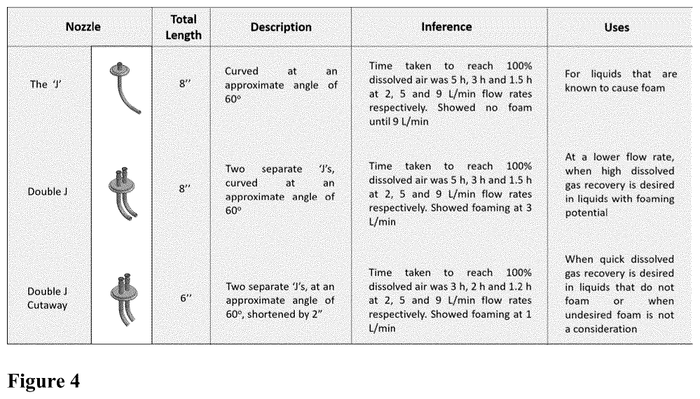

As used herein, the term “nozzle” refers to a pipe or tube of varying cross sectional area, and it can be used to direct or modify the flow of a fluid (liquid or gas). Nozzles are frequently used to control the rate of flow, speed, direction, mass, shape, and/or the pressure of the stream that emerges from them. In a nozzle, the velocity of fluid increases at the expense of its pressure energy. The nozzle may derive from any types, including but not limited to J nozzle, J cutaway nozzle, a double J nozzle, or a double J cutaway nozzle (FIG. 4). The J nozzle is curved to take the shape of the letter ‘J’ and the curve enables directing the flow of the liquid along the wall of the bag to minimize foaming. The double J nozzle is similar to the J, but has two ejector ends as shown in FIG. 4. The double J cutaway replicates the double J nozzle, but is shorter (FIG. 4). Each nozzle is optimal for a different application as outlined in FIG. 4. One of ordinary skill in the art will be aware of, and will be able to choose, suitable nozzle for use in practicing the present invention.

As used herein “bioreactor” takes its art recognized meaning and refers to a chamber designed for the controlled growth of a cell culture. The bioreactor can be of any size as long as it is useful for the culturing of cells, e.g., mammalian cells. Typically, the bioreactor will be at least 30 ml and may be at least 1, 10, 100, 250, 500, 1000, 2500, 5000, 8000, 10,000, 12,0000 liters or more, or any intermediate volume. The internal conditions of the bioreactor, including but not limited to pH and temperature, are typically controlled during the culturing period. A suitable bioreactor may be composed of (i.e., constructed of) any material that is suitable for holding cell cultures suspended in media under the culture conditions and is conductive to cell growth and viability, including glass, plastic or metal; the material(s) should not interfere with expression or stability of a protein of interest. One of ordinary skill in the art will be aware of, and will be able to choose, suitable bioreactors for use in practicing the present invention.

In certain aspects of the invention, the storage container is a bioreactor. In certain aspects of the invention, the storage container is a bag or a tank. In certain aspects of the invention, the bag is a single use bag.

As used herein, a “mixture” comprises a protein of interest (for which purification is desired) and one or more contaminant, i.e., impurities. In one embodiment, the mixture is produced from a host cell or organism that expresses the protein of interest (either naturally or recombinantly). Such mixtures include, for example, cell cultures, cell lysates, and clarified bulk (e.g., clarified cell culture supernatant).

As used herein, the terms “separating” and “purifying” are used interchangeably, and refer to the selective removal of contaminants from a mixture containing a protein of interest (e.g., an antibody).

As used herein, the term “clarified bulk” or “clarified harvest” refers to a mixture from which particulate matter has been substantially removed. Clarified bulk includes cell culture, or cell lysate from which cells or cell debris has been substantially removed by, for example, filtration or centrifugation.

In certain aspects of the invention, the solution is clarified harvest or partially purified protein solution.

As used herein, the term “overlay” refers to a volume of air contained in a container, such as a bag, above a solution comprising a recombinant protein, such as clarified bulk. The air overlay is or is not refreshed over time with new air or a new gas, for example through inlet and outlet ports in the container.

In certain aspects of the invention, the air overlay space comprises greater than 1% of the interior volume of the container.

In certain aspects of the invention, the air overlay space comprises about 5 to 50% of the interior volume of the storage container.

Various aspects of the disclosure are described in further detail in the following subsections.

### Recombinant Polypeptides

The methods of the present invention can be used for large-scale production of any recombinant disulfide bond containing polypeptide of interest, including therapeutic antibodies. Non-limiting examples of recombinant antibodies within the scope of the present invention include, but are not limited to: anti-HER2 antibodies including Trastuzumab (HERCEPTIN®) (Carter et al., Proc. Natl. Acad. Sci. USA, 89:4285-4289 (1992); anti-HER3 antibodies; anti-HER4 antibodies; U.S. Pat. No. 5,725,856); anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108B1, or Tositumomab (BEXXAR®); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN® (Kim et al., Growth Factors, 7:53-64 (1992), International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD11a (U.S. Pat. No. 5,622,700, WO 98/23761, Steppe et al., Transplant Intl. 4:3-7 (1991), and Hourmant et al., Transplantation 58: 377-380 (1994)); anti-IgE (Presta et al., J. Immunol. 151:2623-2632 (1993), and International Publication No. WO 95/19181); anti-CD18 (U.S. Pat. No. 5,622,700, issued Apr. 22, 1997, or as in WO 97/26912, published Jul. 31, 1997); anti-IgE (including E25, E26 and E27; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-Apo-2 receptor antibody (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al., mJ. Immunol. 156 (4): 1646-1653 (1996), and Dhainaut et al., Crit. Care Med. 23 (9): 1461-1469 (1995)); anti-Tissue Factor (TF) (European Patent No. 0 420 937 B1 granted Nov. 9, 1994); anti-human α₄β₇-integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT®) and (ZENAPAX®) (See U.S. Pat. No. 5,693,762 issued Dec. 2, 1997); anti-CD4 antibodies such as the cM-7412 antibody (Choy et al., Arthritis Rheum 39 (1): 52-56 (1996)); anti-CD52 antibodies such as CAMPATH-1H (Riechmann et al., Nature 332:323-337 (1988)); anti-Fc receptor antibodies such as the M22 antibody directed against FcγRI as in Graziano et al., J. Immunol. 155 (10): 4996-5002 (1995); anti-carcinoembryonic antigen (CEA) antibodies such as hMN-14 (Sharkey et al., Cancer Res. 55 (23 Suppl): 5935s-5945s (1995); antibodies directed against breast epithelial cells including huBrE-3, hu-Mc 3 and CHL6 (Ceriani et al., Cancer Res. 55 (23): 5852s-5856s (1995); and Richman et al., Cancer Res. 55 (23 Supp): 5916s-5920s (1995)); antibodies that bind to colon carcinoma cells such as C242 (Litton et al., Eur J. Immunol. 26 (1): 1-9 (1996)); anti-CD38 antibodies, e.g. AT 13/5 (Ellis et al., J. Immunol. 155 (2): 925-937 (1995)); anti-CD33 antibodies such as Hu M195 (Jurcic et al., Cancer Res 55 (23 Suppl): 5908s-5910s (1995) and CMA-676 or CDP771; anti-CD22 antibodies such as LL2 or LymphoCide (Juweid et al., Cancer Res 55 (23 Suppl): 5899s-5907s (1995)); anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-αvβ3 antibody VITAXIN®; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); anti-human leukocyte antigen (HLA) antibodies such as Smart ID10; anti-PD-1 antibodies; anti-PD-L1 antibodies; anti-LAG-3 antibodies; anti-GITR antibodies; anti-TIGIT antibodies; anti-CXCR4 antibodies; anti-CD73 antibodies; anti-IL-13 antibodies, anti-SARS-COV-2 spike protein antibodies and the anti-HLA DR antibody Oncolym (Lym-1).

In certain aspects of the invention, the monoclonal antibody binds an antigen selected from the group consisting of PD-1, PD-L1, CTLA-4, LAG-3, TIGIT, GITR, CXCR4, CD73 HER2, VEGF, CD20, CD40, CD11a, tissue factor (TF), PSCA, IL-8, IL-13, EGFR, SARS-COV-2 spike protein, HER3, and HER4.

In certain aspect of the invention, the recombinant protein is Nivolumab.

### Dissolved Oxygen Control

Disulfide bond reduction is a problem during the protein (mAb) manufacturing and can be minimized to inhibit or decrease the enzyme available to catalyze the disulfide bond reduction reaction (Ren, et al., Biotechnol Bioeng 118 (8): 2829-2844 (2021)) Oxygen can react with NADPH; thus improving DO levels during the harvest can minimize disulfide bond reduction (Handlogten, et al., Biotechnol Bioeng 117 (5): 1329-1336 (2020); Mun et al., Biotechnol Bioeng 112 (4): 734-42 (2015)).

In one embodiment of this invention, a method of controlling the level of a dissolved oxygen (DO) in a solution which comprises a recombinant protein in a storage container, comprising recirculating the solution by drawing the solution to the air overlay of the top of the container, was developed.

In certain aspects of the invention, the recirculating is through a tube. In certain aspects of the invention, the recirculation is driven by a pump. In certain aspects of the invention, the pump is a magnetic pump or a peristaltic pump. In certain aspects of the invention, the recirculated solution flows down along the interior wall of the container.

In certain aspects of the invention, the recirculated solution is directed to the liquid surface or the interior wall of the container. In certain aspects of the invention, the top end of the tube is connected to a nozzle. In certain aspects of the invention, the nozzle is curved. In certain aspects of the invention, the nozzle is a J nozzle, J cutaway nozzle, a double J nozzle, or a double J cutaway nozzle.

In certain aspects of the invention, the solution is recirculated at a flow rate greater than 0.1 L/min. In certain aspects of the invention, the solution is recirculated at a flow rate between 0.1 L/min to 100 L/min.

In certain aspects of the invention, the method further comprises refreshing the air overlay with new air or a new gas through inlet and outlet ports in the container.

In certain aspects of the invention, the new gas is oxygen.

In certain aspects of the invention, the DO in the solution is modified to between 0.00% to 100%.

In certain aspects of the invention, the DO in the solution is increased.

In certain aspects of the invention, the DO in the solution is changed to above 30%.

In certain aspects of the invention, the DO in the solution is changed to above 30%, above 40%, above 50%, above 60%, above 70%, above 80%, above 90%, or about 100%.

Proteins are also susceptible to oxidative damage through reaction of certain amino acids (i.e. methionine, cysteine, histidine, tryptophan, and tyrosine) with oxygen radicals in their environment, and the oxidation of protein can result in protein degradation. Oxidation can be induced during protein processing and storage (Patel et al., BioProcess International 9 (1): 20-31 (2011)).

In certain aspects of the invention, the new gas is carbon dioxide.

In certain aspects of the invention, the new gas is an inert gas. In certain aspects of the invention, the new gas is nitrogen or argon.

In certain aspects of the invention, the dissolved oxygen level in the solution is decreased.

In certain aspects of the invention, the DO in the solution is changed to less than 30%.

In certain aspects of the invention, the DO in the solution is changed to below 30%, below 25%, below 20%, below 15%, below 10%, below 5%, below 1%, or about 0.00%.

The foregoing description is to be understood as being representative only and is not intended to be limiting. Alternative methods and materials for implementing the invention and also additional applications will be apparent to one of skill in the art, and are intended to be included within the accompanying claims.

### Example

### Set Up and Assembly

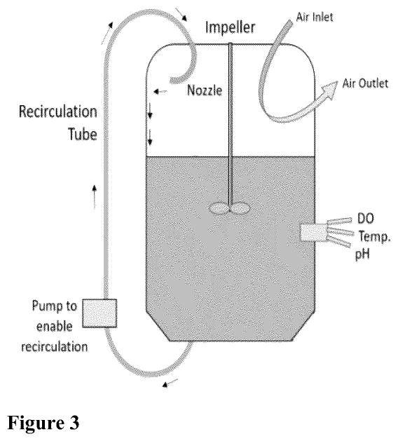

The assembly consisted of a polypropylene nozzle within a single-use recirculation bag which was setup in a single use mixer of any size depending on process requirements. The bag was a modified version of an industry standard powder port bag with the provision of a ¾″ O. D.×½″ I. D. tube originating from the bottom of the bag and extending up to the top. This served as the recirculation tube. The tube was connected to the top of the bag at the powder port. The port connection consisted of a nozzle (R&D Enterprises) as shown in FIG. 4. The connection between the nozzle and the bag was made at the powder port connector. A ‘J’ shaped nozzle was connected to the bag through the powder port and the recirculation tube was connected to the nozzle. As demonstrated in FIG. 3, the nozzle was curved towards the wall of the bag ensuring that the liquid flowed downward against the wall, rather than falling vertically into the liquid, creating splashing. Using the current design, splashing was prevented, minimizing foaming. The single-use bag had 2 vent filters, Pall Kleenpak air filters (KA3V002PVIG), which were located at the top of the bag. One vent filter was the source for gas into the headspace and the second vent filter is to facilitate the exit of gas. Mettler Toledo inline dissolved oxygen and pH probes (6860i, Inpro3253i) were used to detect dissolved oxygen, pH, and ORP levels in the system.

### Nozzle Variability

Several different types of nozzles were used for the recirculation, besides the J, as shown in FIG. 4. The J nozzle was curved to take the shape of the letter ‘J’. The curve enabled directing the flow of the liquid along the wall of the bag to minimize foaming. The double J nozzle was similar to the J, but had two ejector ends as shown in FIG. 4. The double J cutaway replicated the double J nozzle, but was shortened by 1.5 inches (FIG. 4). Each nozzle was optimal for a different application as outlined in FIG. 4. The recirculation bag was designed to accommodate nozzles of any size, shape or design. The nozzles explained in this application had three particular examples.

### Recirculation

A Levitronix pump (PuraLev i100SU) was used to induce recirculation from the bottom of the system to the top (FIG. 3). The Levitronix pump utilized magnetic levitation instead of peristaltic motion, thus reducing mechanical sheer of the liquid constituents.

Sparger's Effectiveness in Dissolved Oxygen Recovery in Comparison with the Air Overlay Method and Static Air

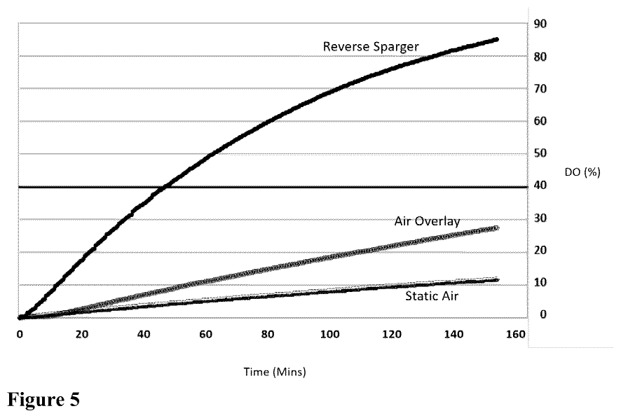

FIG. 5 shows the results of an experiment to compare the reverse sparger's effectiveness in dissolved oxygen recovery in comparison with the air overlay method and static air (no inlets or outlets). Dissolved oxygen recovery experiments were performed for the reverse sparger vessel, air overlay vessel, and static air vessel. Dissolved oxygen recovery is a common experiment in which the dissolved oxygen is reduced to 0%. This was achieved by a method known as the dynamic gassing out method where nitrogen gas was introduced in the vessels, depleting the oxygen. The supply of nitrogen was terminated when the dissolved oxygen reaches the desired value (0%, in this case). Once the dissolved oxygen (DO) was at 0%, oxygen was supplied into the vessels. The DO content thus continuously increased until at a certain point it stabilized. The vessel which reached the highest dissolved oxygen percentage (starting from 0) in the shortest period of time (for example, 3 hours) was the vessel which had the highest dissolved oxygen recovery rate.

The experiment was performed on the three conditions described above, the reverse sparger, air overlay and static air. In the experiment each vessel contained 350 L of phosphate buffer solution (PBS) at 2-8° C. The reverse sparger liquid flow rate used was 5 L/min and had the fastest recovery rate, as demonstrated in FIG. 5. Over a three-hour period the reverse sparger reached 85% DO whereas the air overlay system only reached 27% DO. Studies have reported the formation of Low Molecular Weight (LMW) species can occur within hours of harvest operations and a dissolved oxygen content of below 30% (Mun et. al., Biotechnology and Bioengineering 112, 734-742 (2015)). During the experiments a DO concentration of 40% was targeted to ensure DO concentration was maintained above 30%. The reverse sparger achieved 40% DO in approximately 48 minutes compared to the air overlay which as unable to reach 40% in three hours, and the static air system which reached approximately 12% DO in three hours. This demonstrates the reverse sparger has the potential to be a more effective method to minimize LMW formation and prevent protein degradation.

### Reverse Sparger's Effectiveness in DO Control

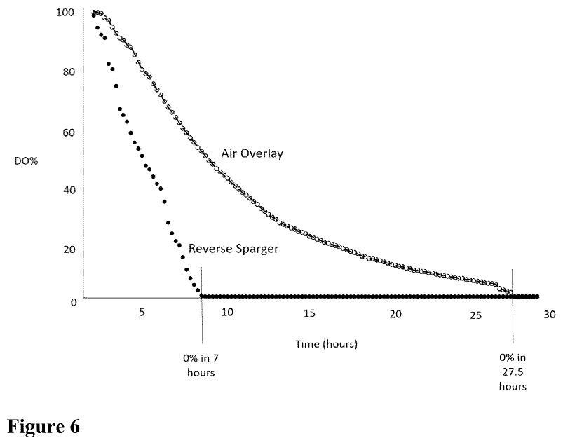

FIG. 6 shows the results to demonstrate the reverse sparger's effectiveness in DO control. Several mAbs had processing requirements where the DO was required to be reduced from one set point to another. The reverse sparger can achieve this at a rate faster than that of the air overlay system. The methods of this experiment were similar to the methods used to conduct the above experiment, but in reversed order. The vessels contained 350 L of PBS at 2-8° C. Air was continuously added to the vessels until the DO stabilized at 100%. Both systems were then supplied with nitrogen gas which was used to reduce the DO content by depleting the oxygen. The reverse sparger reduced the DO content quicker than air overlay system, reaching 0% DO within 7 hours and 27.5 hours respectively.

### Comparison of the k_{L}a Among the Reverse Sparger and the Industry Standard Spargers

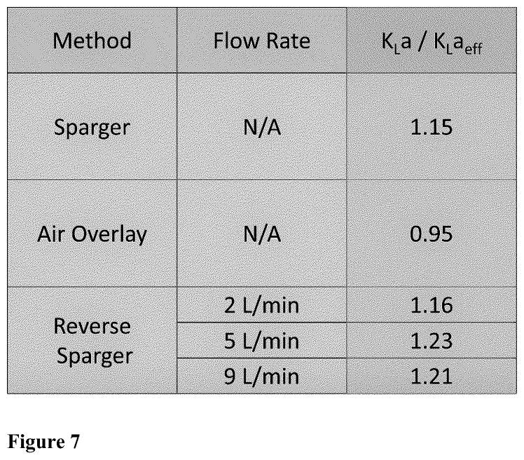

FIG. 7 compares the k_{L}a values of the reverse sparger with the industry standard sparger. This is a quantitative method typically used to compare the effectiveness of different gas mass transfer methods (Arrua et al., AlChE J 36:1768-1772 (1990)). The k_{L}a values of oxygen transfer provide quantitative information on the rate at which oxygen is transported to the liquid from the gaseous phase. K_{L}a values were calculated to compare the oxygen transfer rate of the reverse sparger with the sparger. The dynamic gassing out method was used to obtain values.

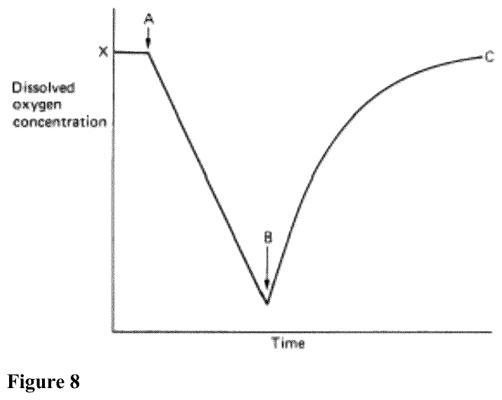

Nitrogen gas was used to deplete oxygen from the liquid until the dissolved oxygen level was down to 0% in all three conditions (dynamic overlay, sparger and the reverse sparger). Oxygen was then introduced in each of the systems (FIG. 8). The k_{L}a method is typically used for cell culture based bioreactor operations which contain oxygen consuming media. The reverse sparger gassing out method was performed with non-oxygen consuming media, hence the value ‘k_{L}a_{eff}’ was introduced. The slope of the oxygen recovery graph was used to obtain the k_{L}a and k_{L}a_{eff} values using the formulas, k_{L}a=slope ln (C_{L0}-C_{L}) and k_{L}a_{eff}=slope ln (C_{L0}-C_{L}). Where k_{L}a is defined as volumetric oxygen mass transfer coefficient, C_{L0} is the initial dissolved oxygen concentration, and C_{L} is dissolved oxygen concentration. Similar to traditional k_{L}a values, the k_{L}a_{eff} values are a measure of sparging effectiveness with a higher number representing better mass transfer. The k_{L}a_{eff} values of the reverse sparger were calculated for all three nozzles, at flow rates of 2 L/min, 5 L/min and 9 L/min. As seen in FIG. 7, the k_{L}a value of the open pipe sparger was 1.15 h⁻¹. The k_{L}a_{eff} values for all three nozzles used were in the range of 1.11 to 1.23 h⁻¹ at all three flow rates. These values were highly comparable with the k_{L}a value obtained for the sparger experiment that was run in parallel. This confirmed the comparability of the reverse sparger to that of the industry standard sparger. The air overlay had a k_{L}a value of 0.95 h⁻¹ which was lower than the k_{L}a_{eff} values of the reverse sparger and the k_{L}a value of the sparger.

### Reverse Sparger Study

TABLE 1
Quality data of samples taken on Day 1, 2, 4, and
7 of continuous reverse sparging in a bag holding
clarified bulk (CB) containing a mAb at 2-8° C.

| | | Clarified | Reverse | Reverse | Reverse | Reverse |
|---|---|---|---|---|---|---|
| | | Bulk | Sparger | Sparger | Sparger | Sparger |
| Assay | Sample | Day 0 | Day 1 | Day 2 | Day 4 | Day 7 |
| N-glycan | G0-GN | 0.2 | 0.2 | 0.2 | 0.2 | 0.2 |
| | G0F-GN | 0.7 | 0.6 | 0.7 | 0.7 | 0.7 |
| | G0 | 1.1 | 1.1 | 1.1 | 1.1 | 1.1 |
| | G0F | 54.9 | 54.2 | 54.2 | 53.7 | 54.0 |
| | Man5 | 1.4 | 1.3 | 1.4 | 1.3 | 1.3 |
| | G1 | 0.5 | 0.4 | 0.4 | 0.4 | 0.4 |
| | G1′ | 0.3 | 0.3 | 0.2 | 0.2 | 0.2 |
| | G0FN | 0.5 | 0.5 | 0.5 | 0.5 | 0.5 |
| | G1F | 19.5 | 19.7 | 20.3 | 20.7 | 20.8 |
| | G1F′ | 13.1 | 13.2 | 12.2 | 12.1 | 12.0 |
| | G1FN | 1.5 | 0.7 | 0.8 | 0.7 | 0.8 |
| | G1FS1 | | 0.8 | 0.7 | 0.7 | 0.7 |
| | G2F | 5.9 | 6.1 | 6.0 | 6.1 | 6.1 |
| Size- | Monomer | 97.9 | 98.0 | 97.9 | 98.2 | 98.4 |
| Exclusion | HMW | 0.7 | 0.7 | 0.8 | 0.5 | 0.3 |
| Chroma- | LMW | 0.7 | 0.8 | 0.7 | 0.7 | 0.6 |
| tography | | | | | | |
| Imaged | Acidic | 56.1 | 56.6 | 57.3 | 57.7 | 56.7 |
| Capillary | (%) | | | | | |
| Isoelectric | Basic (%) | 3.3 | 3.2 | 3.0 | 2.9 | 2.8 |
| Focusing | Main | 40.6 | 40.2 | 39.7 | 39.4 | 40.4 |
| | Peak (%) | | | | | |
| Caliper- | Relative | 95.7 | 97.4 | n/a | 99.6 | n/a |
| NR | Purity | | | | | |

Table 1 shows the quality data of samples taken on Day 1, 2, 4, and 7 of continuous reverse sparging in a bag holding clarified bulk (CB) containing a mAb at 2-8° C. The data in FIG. 9 shows the N-glycan, SEC, and iCHEF, and caliper-NR results from the reverse sparger, as well as the Day 0 CB material. The results suggested that the continuous operation of the reverse sparger did not have a major negative impact to protein quality for at least 7 days.

1. A method of controlling the level of a dissolved oxygen (DO) in a solution which comprises a recombinant protein in a storage container, comprising recirculating the solution by drawing the solution to the air overlay of the top of the container.

2. The method of claim 1, wherein the recirculating is through a tube.

3. The method of claim 1, wherein the recirculation is driven by a pump.

4. The method of claim 1, wherein the recirculated solution is directed to the liquid surface or to the interior wall of the container

5. The method of claim 2, wherein the top end of the tube is connected to a nozzle.

6. The method of claim 5, wherein the nozzle is curved.

7. The method of claim 6, wherein the nozzle is a J nozzle, J cutaway nozzle, a double J nozzle, or a double J cutaway nozzle.

8. The method of claim 1, wherein the air overlay space comprises greater than 1% of the interior volume of the storage container.

9. The method of claim 1, further comprising refreshing the air overlay with new air or a new gas through inlet and outlet ports in the container.

10. The method of claim 9, wherein the new gas is oxygen.

11. The method of claim 9, wherein the new gas is nitrogen, argon or carbon dioxide.

12. The method of claim 11, wherein the new gas is nitrogen.

13. The method of claim 1, wherein the storage container is a bag or a tank.

14. The method of claim 1, wherein the recombinant protein is a recombinant disulfide-bond containing protein.

15. The method of claim 14, wherein the recombinant disulfide-bond containing protein is a monoclonal antibody.

16. The method of claim 15, wherein the monoclonal antibody binds an antigen selected from the group consisting of PD-1, PD-L1, CTLA-4, LAG-3, TIGIT, GITR, CXCR4, CD73, HER2, VEGF, CD20, CD40, CD11a, tissue factor (TF), PSCA, IL-8, IL-13, EGFR, SARS-COV-2 spike protein, HER3, and HER4.

17. The method of claim 14, wherein the recombinant disulfide-bond containing protein is a Fc-fusion protein.

18. The method of claim 1, wherein the solution is a clarified bulk or a partially purified protein solution.
